(19) United States
(12) Patent Application Publication
(10) Pub. No.: US 2005/0106656 A1
(43) Pub. Date: May 19, 2005

(54) Semi-automated microscopic assessment of islet quantity, equivalence and purity

(76) Inventor: Ingrid Stuiver, San Diego, CA (US)

Correspondence Address: GORDON & REES LLP, 101 WEST BROADWAY, SUITE 1600, SAN DIEGO, CA, 92101, US

(21) Appl. No.: 10/956,293

(22) Filed: Oct. 1, 2004

Related U.S. Application Data
(60) Provisional application No. 60/507,780, filed on Oct. 1, 2003.

(51) Int. Cl. G06F 19/00; G01N 33/48; G01N 33/50; G01N 1/30; G06K 9/00

(52) U.S. Cl. 435/40.5; 382/128; 702/19

(57) ABSTRACT

A method of quantifying islets of Langerhans via rapid microscopic image analysis.

This application is based on U.S. Provisional Application Ser. No. 60/507,780 filed Oct. 1, 2003.

### FIELD OF THE INVENTION

The present invention is directed toward semi-automated islet quantification.

### BACKGROUND

Islets have been quantified using a Coulter Multisize Ile which is used to simply measure particle size. Integrity and purity of islets cannot be measured with this equipment. The present invention addresses both of these issues using digital image analysis with a black and white CCD camera where particle size can be counted, but not purity and integrity of the islets.

### DESCRIPTION OF THE INVENTION/SOLUTION

A system has been developed that can semi-automatically quantify islets of Langerhans via rapid microscopic image analysis (Compix, Inc.). The exact area of the particle is determined, the average diameter of this area obtained and used to convert to the volume of a sphere. This volume is then converted to the current standard of islet quantification, the islet equivalence (IE). One IE is equivalent to an islet of 150 μm in diameter. The IE of each particle that is 13 μm or larger (due to the limitations of the lens used in our set-up) contained within a well is calculated. Multiple samples can be quantified at once (96 well plate in 5 minutes) with minimal sample to sample variability. The measurement of optimal saturation levels for identification of each object is the only area that is operator-dependent. This method of quantification can be adapted to several available software systems such as Simple PCI and Image Pro. This process enables the rapid quantification and qualification of islets, by size, number and purity.

### TECHNICAL/EXPERIMENTAL DESCRIPTION OF THE INVENTION

Microscope

Nikon TE-2000S equipped with a motorized-x-y-z stage and a side-mounted CCD camera.

Digital Camera

Spot RT Slider—color CDD Camera

Software

SimplePCI, AIC and IPA Modules (Compix, Inc.). SimplePCI allows image enhancements, presentations and analysis operations to be easily applied to single images or image sequences. SimplePCI is a high-performance imaging application, designed to meet the need for high-speed processing at a low cost, with ease of use and flexibility. SimplePCI is optimized to use Intel's MMX instructions, accelerating imaging operations by 3 to 5 times. At the core of SimplePCI are the Image Documents and Data Documents, allowing image enhancements, presentation and analysis operations to be easily applied to single images or image sequences; and allowing threshold objects or manual drawing of multiple regions for analysis, and then present the data as a list, statistical summary, or histogram. Field analysis of the data provides both summary and individual region information.

The Automated Image Capture (AIC) module enhances SimplePCI to provide image capture from a wide array of cameras. The Image Processing and Analysis (IPA) enable the development of icon driven work-files (macros) for multiple application solutions. Similar profiles can be obtained using Image Pro. In fact, Image Pro has a preprogrammed 96 well plate that one can select specific wells to quantify the image(s).

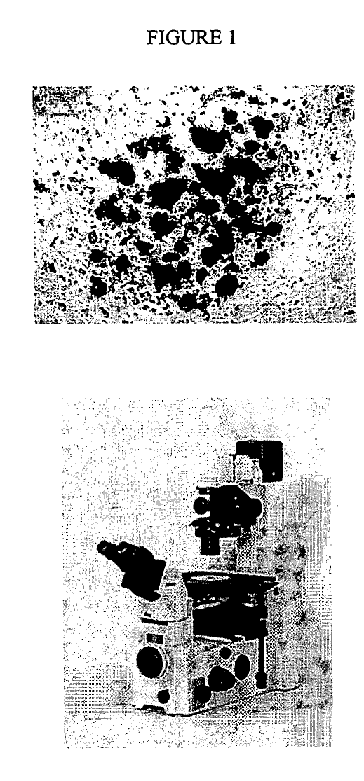

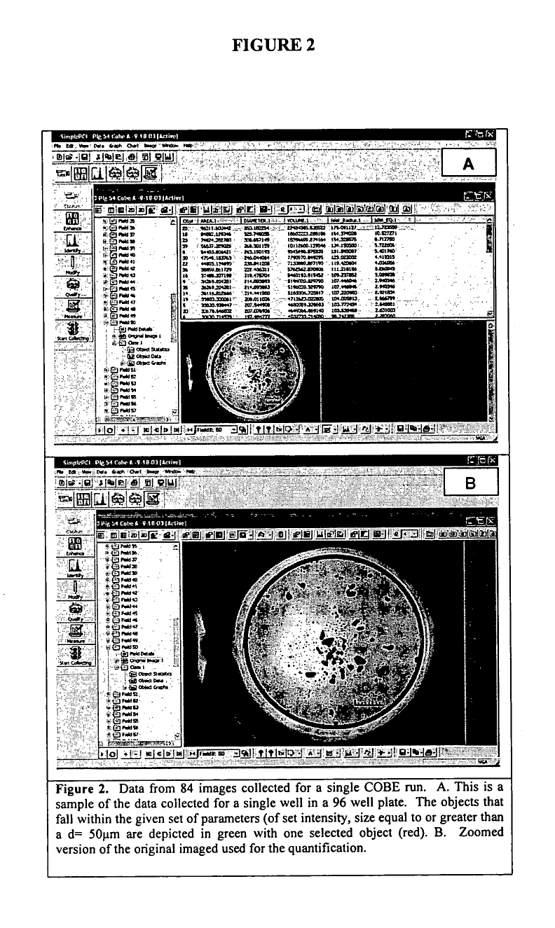

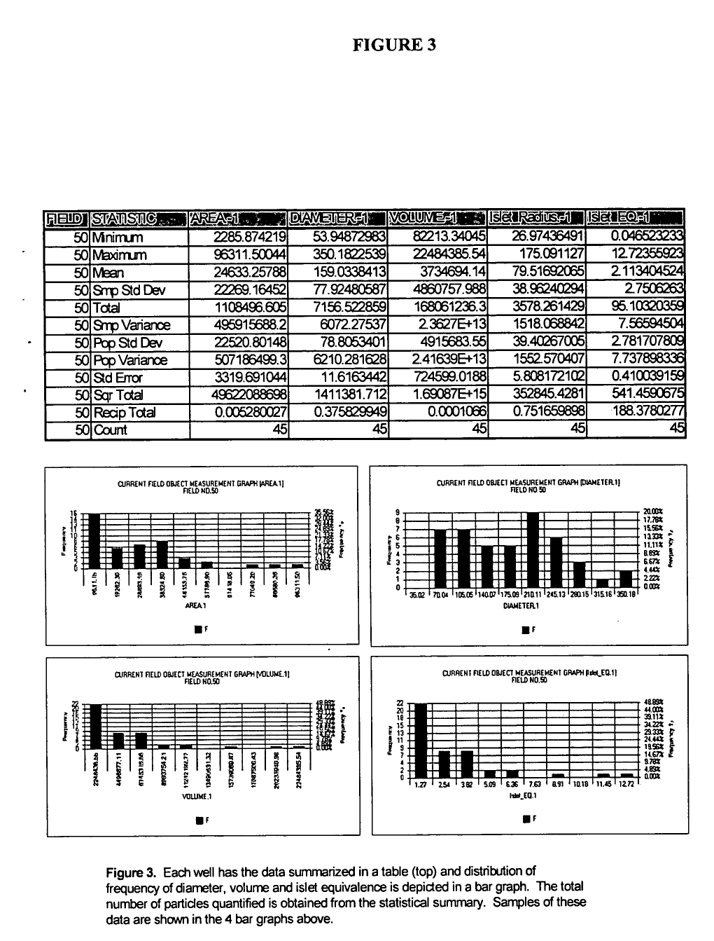

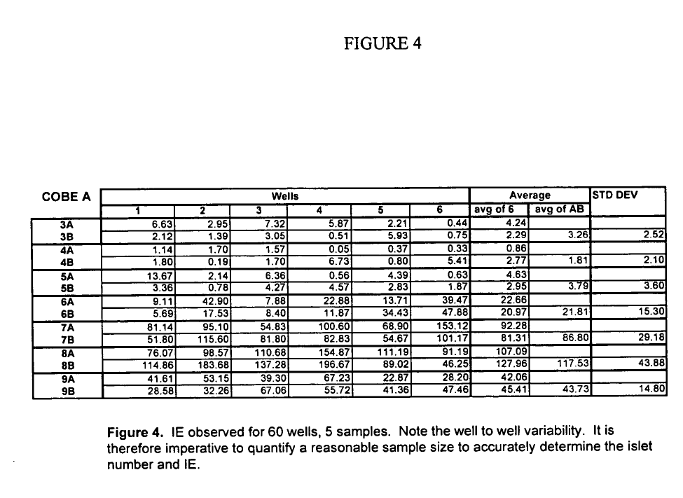

### FIG. 1.

A. Porcine islets stained with Dithizone B. Zeiss TE 2000S.

### METHODS

Calibration

The system is calibrated using 149 micron red particles made of polyacrylamide. Up to 50 particles are placed in 2 wells of a 96-well plate set in the microscope stage. The system can be calibrated at a set light intensity by performing the “white balance function” of the Spot Camera software. The system calibrates to a consistent image exposure for that light intensity. This function minimizes user variability in obtaining result, something frequently seen when counting manually. When the lighting has been set, an image is captured. The program is calibrated by running through each step of the program: The image is lightened followed by an adjustment of the HLS (Hue, Lightness, Saturation transform) such that the “region of interest” (ROI) is set to select the particles of interest. The hue and saturation are adjusted so that the complete perimeter of the islet or particle is captured. These particles are then qualified by diameter, area, roundness and vector center. The objects that are too large in diameter are excluded followed by the programmable quantification and characterization of the particles in each well. These parameters can be user defined. A typical calibration profile measures bead sizes in the range of 139-144 microns in size. The resulting IEs approximate 1. These results indicate that the system can accurately measure particle size.

Image Capture and Processing

The SPOT RT slider camera used is a color camera. The color image of the well is captured and this image is used for particle identification. A program written using the IPA module (use of customized icon driven macros) from Simple PCI (Compix Inc., Imaging Systems) calculates the size, qualifies and quantifies the number of particles/islets based on saturation levels of each particle. The number of pixels above a set intensity threshold of the RGB (Red, Green, Blue) value allows for the discrimination of the dithizone stained islets. Each particle that is identified by these pixels is qualified based on linearity, size of area and shape. The exact area of each qualified particle is then determined. From this, the mean diameter of each particle is calculated followed by its conversion to volume and subsequently to IE. Islet equivalence is determined by the following formula:
IE of particle=Volume of observed sphere/Volume of a 150 micron diameter islet

Image capture, enhancement and quantification parameters; image enhancement is used to brighten the captured image, the pixel saturation set so that each object is identified. The objects are qualified by area, diameter, volume. Those that meet these qualifications are quantified.

The computer program (a series of macros) was written to analyze the image and determine the number of qualified islets, followed by the calculation of IE. The formula provided above was used and the Simple PCI software enabled the development of icon driven work-files (macros) for this particular application.

Quantification

Samples of statistically relevant amounts are provided and islets stained in a homogeneous solution containing dithizone or any preferred stain are disbursed into wells of a 96-well plate. The well is topped off with a total final volume of 360 μl so that the meniscus does not interfere with image capture. Once the image of the well is captured all particles selected are instantly analyzed for area and the programmed calculations performed. The final desired output in this instance is islet equivalence. Each plate is processed within 3 minutes.

This system addresses current problems with manual islet quantification. They are: Estimation of islet volume in 2 dimensions which can lead to over or under estimations of IE, small sample volume limiting the accuracy of total number of islets and IE; time required to count multiple samples; limited reproducibility and accuracy due to operator to operator variability.

This system is being extended to quantify islets followed by quantification of exocrine tissue that may be contaminating the sample. The values obtained will then be used to determine the purity in each well. 3-dimensional image analysis is also being considered as a means to more accurately quantify islet mass.

The system described here uses a color CCD camera so it is possible to discriminate between islet and exocrine tissue and assess the purity in addition to quantification of the particles in each well. The motorized stage enables quantification of large numbers of samples (96 per plate), thus increasing accuracy and output and decreasing the time it takes to quantify multiple samples by approximately 10 fold. Previously, manual counting of 20 samples took 5 hours. With this system, digital records of quantity, integrity and purity of 96 samples are obtained within 5 minutes.

The present invention, compared to prior methods, provides a structural distinction, namely: Color CCD Camera, xyz motorized stage, high quality 2× lens, Simple PCI systems program, 96 well plate counting capabilities, macro-driven computer program, and the use of standardized beads to calibrate the system. This invention involves a scalable procedure. Other methods in the industry were aimed at a rapid measure to quantify islets, but not necessarily to evaluate the integrity of the islet. The present invention allows the operator to do both.

For biomedical/pharmaceutical development, and for clinical therapeutics or diagnostics, the present invention enables the rapid quantification and qualification of islets, by size, number and purity. This process is scalable.

References and Patents (Herein Incorporated by Reference)

Lehmann, R., et al., Evaluation of islet isolation by a new automated method (Coulter Multisizer Ile) and manual counting. Transplant Proc, 1998. 30(2): p. 373-4.

Stegemann, J. P., et al., Improved assessment of isolated islet tissue volume using digital image analysis. Cell Transplant, 1998. 7(5): p. 469-78.

Ricordi, C., et al., Pancreatic Islet Cell Transplantation. Islet Isolation Assessment, ed. C. Ricordi. Vol. 1892-1992. 1992, Pittsburgh, Pa.: R. G. Landes Company. 132-142

Gayler, J. & Burnip, B. High Res Image Analysis for NIH “Civilians”. Adv. Imag. February 34-38 (1992).

Compix Inc., Imaging Systems, was incorporated in the State of Pennsylvania in 1987 by Bill Burnip and John Gayler. On Feb. 5, 2003—Compix Inc., Imaging Systems was acquired by Hamamatsu Photonics Management Corporation, Del.

References cited herein are, to the extent appropriate, incorporated by reference to supplement this disclosure.

It will be understood that one of skill in the art is not limited to the cameras, microscopes, and software disclosed herein, but can easily determine which cameras, microscopes and software are commercially available as substitutes to achieve the objectives of the invention.

1. A method of quantifying islets of Langerhans via rapid microscopic image analysis, comprising the steps of:
a. determining the exact area of a particle,
b. determining the average diameter of the areas obtained,
c. converting the average diameter measurement into spherical volume, and
d. converting said spherical volume measurement into islet equivalence.
